(19) United States
(12) Patent Application Publication
(10) Pub. No.: US 2006/0228735 A1
(43) Pub. Date: Oct. 12, 2006

(54) Multiplex assay systems

(75) Inventors: Mark N. Bobrow, Lexington, MA (US); Mack J. Schermer, Belmont, MA (US)

Correspondence Address: FISH & RICHARDSON PC, P.O. BOX 1022, MINNEAPOLIS, MN, 55440-1022, US

(73) Assignee: PerkinElmer LAS, Inc.

(21) Appl. No.: 11/394,673

(22) Filed: Mar. 31, 2006

Related U.S. Application Data
(60) Provisional application No. 60/668,769, filed on Apr. 6, 2005.
(60) Provisional application No. 60/666,910, filed on Mar. 31, 2005.

(51) Int. Cl. C12Q 1/68; C12M 1/34

(52) U.S. Cl. 435/6; 435/287.2

(57) ABSTRACT

The methods described herein can be used, e.g., for detecting at least two target molecules in a sample (e.g., a single sample). Exemplary methods include using a target-specific switch probe attached to a substrate and using zip codes to associate holographically encoded particles with a target-specific probe.

### CROSS-REFERENCE TO RELATED APPLICATIONS

This application claims priority to U.S. Applications Ser. No. 60/668,769, filed on Mar. 31, 2005 and 60/666,910, filed Apr. 6, 2005, the contents of each of which are hereby incorporated by reference in their entireties.

### BACKGROUND

Probes can be used to evaluate the molecular contents of a sample. For example, oligonucleotides can be used to detect specific nucleic acids in a sample. Antibodies can be used to detect any of a broad range of antigens.

### SUMMARY

In one aspect, the disclosure features a solid substrate that has attached to it a target-specific switch probe. In some embodiments, the substrate is a particle such as an encoded particle, e.g., an optically encoded. An example of an optically encoded particle is a holographically encoded particle. In some embodiments, the substrate includes an array. For example, the substrate has a surface, such as a planar surface. The surface can include a plurality of positionally distinguishable addresses. The target-specific switch probe is attached to at least one of the addresses. Multiple different target-specific switch probes can be attached to the substrate. For example, each of the plurality of positionally distinguishable addresses includes a different target-specific switch probe.

In some embodiments, the target-specific switch probe produces a fluorescent signal that varies depending on whether the target molecule, for which the target-specific switch probe is specific, is present. For example, the target-specific switch probe includes a fluorescer and a quencher, and the distance between the fluorescer and the quencher is increased when the target specific switch probe hybridizes to the target.

In some embodiments, the target specific switch probe includes a primer sequence. For example, the target specific switch probe includes a first strand that include a fluorescer, a probe region, a blocker, and a primer, and a second strand that includes a quencher that is attached to quencher stand that hybridizes to the probe region unless the primer is extended, and wherein the primer is specific for a target nucleic acid sequence and is extendable to include a sequence complementary to the probe region.

In some cases, the target specific switch probe can form a hairpin under particular conditions in the absence of the target. In other cases, the target specific switch probe can form a hairpin under particular conditions only if it has been contacted to the target, e.g., if a primer sequence of the target specific switch probe has been extended.

In another aspect, the disclosure features a collection of particles. Each particle can have the features of a particle described herein. For example, the collection includes particles having target-specific switch probes specific for different target sequences. In cases where the particles are coded, e.g., optically coded, particles having the same target-specific switch probe have the same code, e.g., the same optical code.

In another aspect, the disclosure features a method of evaluating a plurality of target molecules. The method includes: providing (i) a plurality of target-specific switch probes specific for different target molecules, wherein the target-specific switch probes are physically associated with a substrate and (ii) a sample; and detecting signals associated with each of the different target specific switch probes, thereby detecting target molecules based on interaction between the target-specific switch probes and the respective target molecules for which they are specific.

For example, the substrate includes an array having a plurality of positionally distinguishable areas. Each target-specific switch probe can be physically associated with a different positionally distinguishable area. In other examples, the substrate is a particle, e.g., an encoded particle such as an optically encoded particle. Each target-specific switch probe can be associated with a different particle. The probe can by associated with the particle, before, during, or after the probe is contacted to the sample.

In still another aspect, the disclosure features a method of evaluating a plurality of target molecules, such as nucleic acids, proteins, or small molecules. The method includes: providing a mixture including: (i) encoded particles, wherein each particle having a particular code includes a unique target-specific switch probe for detecting an target molecule, and (ii) a sample; maintaining the mixture under conditions that permit probes of the particles to interact with target molecules in the sample material; and detecting signals associated with each of the encoded particles, thereby detecting interactions between the unique probe of each of the encoded particles and the target molecule that it detects. For example, the encoded particles are optically encoded, e.g., holographically encoded.

In some cases, the target specific switch probe is physically associated with the particle by hybridization to a bridging oligonucleotide covalently attached to the particle. The mixture can include multiple copies of at least some the encoded particles, e.g., at least 3, 5, 10, 20, or 50 copies, e.g., between 2-20 copies. The mixture can include at least 10, 100, 200, or 500 differently encoded particles.

The mixture can be disposed in a well of multi-well plate or in a tube, e.g., a closed tube. The signals associated with each of the encoded particles can be detected using a detector, e.g., a detector that includes a flow cell through which the encoded particles can pass.

In some embodiments, the target specific switch probe is associated to the particle by binding of a capture moiety to a complementary moiety covalently attached to the particle.

In some embodiments, the target-specific switch probe produces a fluorescent signal that varies depending on whether the target molecule, for which the target-specific switch probe is specific, is present. For example, the target-specific switch probe includes a fluorescer and a quencher, and the distance between the fluorescer and the quencher is increased when the target specific switch probe hybridizes to the target.

In some embodiments, the target specific switch probe includes a primer sequence. For example, the target specific switch probe includes a first strand that include a fluorescer, a probe region, a blocker, and a primer, and a second strand that includes a quencher that is attached to quencher stand that hybridizes to the probe region unless the primer is extended, and wherein the primer is specific for a target nucleic acid sequence and is extendable to include a sequence complementary to the probe region.

In some cases, the target specific switch probe can form a hairpin under particular conditions in the absence of the target. In other cases, the target specific switch probe can form a hairpin under particular conditions only if it has been contacted to the target, e.g., if a primer sequence of the target specific switch probe has been extended.

In another aspect, the disclosure features a particle that includes holographically coded information that identifies the particle and a capture moiety, including an oligonucleotide, and a target-specific probe, e.g., a target-specific switch probe. The target-specific probe is associated with the particle by hybridization of a region of the oligonucleotide to a complementary region of the target-specific probe. For example, the region of the oligonucleotide that hybridizes to the target-specific probe is absent from the sample of interest.

In yet another aspect, the disclosure features a set of particles, wherein each particle includes holographically coded information that identifies the particle and a unique capture moiety identifiable by the holographically coded information. The particles can one or more of the following features: (i) the unique capture moiety specifically interacts with a complementary moiety that can be attached to a target specific probe, (ii) the unique capture moiety does not substantially interact with the sample of interest, and (iii) the unique capture moiety does not specifically interacts with the other complementary moieties of other particles in the set.

In another aspect, the disclosure features a method of evaluating a plurality of target molecules. The method includes:

providing a mixture including: (i) holographically encoded particles, wherein each particle having a particular code includes a unique target specific probe for detecting a target molecule, wherein the target specific probe is associated with the particle by a capture moiety covalently attached to the particle and a complementary moiety covalently attached to the target specific probe, and (ii) sample;

maintaining the mixture under conditions that permit probes of the particles to interact with target molecules in the sample to form encoded particle-target molecule complexes; and

detecting signals associated with each of the encoded particle-target molecule complexes, thereby detecting interactions between the unique target-specific probe of each of the encoded particles and the target molecule that it detects. The method can include other features described herein.

In another aspect, the disclosure features a method of evaluating a plurality of target molecules. The method includes: providing a mixture including: (i) unique target-specific probes for detecting different target molecules, and (ii) sample material; maintaining the mixture under conditions that permit the unique target-specific probes to interact with target molecules in the sample material; associating the unique probes with holographically encoded particles, such that each encoded particle specifically associates with replicates of a particular unique target-specific probe; and detecting signals associated with each of the encoded particles, thereby detecting interactions between the unique probe of each of the encoded particles and the target molecule that it detects. The method can include other features described herein. Examples of the target-specific probes include oligonucleotides and antibodies.

For example, each encoded particle is specifically associated with a particular unique probe by interaction between a capture moiety covalently attached to each encoded particle and a complementary moiety covalently attached to a target-specific probe.

In one embodiment, the capture moiety includes an oligonucleotide with a region that is complementary to a sequence that constitutes the complementary moiety of the target specific probe.

The mixture can include multiple copies of at least some the encoded particles, e.g., at least 3, 5, 10, 20, or 50 copies, e.g., between 2-20 copies. The mixture can include at least 10, 100, 200, or 500 differently encoded particles.

The mixture can be disposed in a well of multi-well plate or in a tube, e.g., a closed tube.

In some embodiments, the signals are generated by fluorescent nucleotides incorporated during DNA replication by extension of the unique probes. For example, the fluorescent nucleotides are labeled dideoxy nucleotides. Each nucleotide can include an optically distinguishable label. The signals associated with each of the encoded particles can be detected using a detector, e.g., a detector that includes a flow cell through which the encoded particles can pass.

The method can further include amplifying nucleic acids in the mixture using the target specific probes as primers for extension of target nucleic acid molecules.

In some embodiments, the target-specific probe is a target specific switch probe. In some embodiments, the target-specific switch probe produces a fluorescent signal that varies depending on whether the target molecule, for which the target-specific switch probe is specific, is present. For example, the target-specific switch probe includes a fluorescer and a quencher, and the distance between the fluorescer and the quencher is increased when the target specific switch probe hybridizes to the target.

In some embodiments, the target specific switch probe includes a primer sequence. For example, the target specific switch probe includes a first strand that include a fluorescer, a probe region, a blocker, and a primer, and a second strand that includes a quencher that is attached to quencher stand that hybridizes to the probe region unless the primer is extended, and wherein the primer is specific for a target nucleic acid sequence and is extendable to include a sequence complementary to the probe region.

In some cases, the target specific switch probe can form a hairpin under particular conditions in the absence of the target. In other cases, the target specific switch probe can form a hairpin under particular conditions only if it has been contacted to the target, e.g., if a primer sequence of the target specific switch probe has been extended.

In still another, the disclosure features a method for interrogating nucleic acid sequences in a sample. The method includes:
(a) providing a plurality of encoded particles, wherein each particle having a given code is associated with or can be associated with a target-specific probe;
(b) contacting a sample to the target specific probes;
(c) maintaining the mixture under conditions in which the target specific probes interact with target nucleic acids in the sample, if present; and
(d) associating each of the encoded particles with its respective target-specific probe either before step (b), after (b), or after (c).
(e) evaluating the interactions by detecting the code from each encoded particle and associated information about interaction with the target nucleic acids.

For example, each encoded particle specifically associates with a particular target-specific probe by interaction between a capture moiety covalently attached to each encoded particle and a complementary moiety covalently attached to a target-specific probe. It is possible to use at least 10, 100, 200, or 500 uniquely encoded particles. There may be multiple replicates of a particle have a particular code.

Other embodiments and details are provided herein below.

### BRIEF DESCRIPTION OF DRAWINGS

FIG. 1 is a process flow diagram showing preparation of a holographically encoded particle set and method for performing a multiplexed assay according to an exemplary embodiment.

FIG. 2 depicts a holographically encoded particle.

FIG. 3 depicts a holographically encoded particle having target-specific switch probes immobilized on its surface.

FIG. 4 depicts a magnified view of the particle shown in FIG. 3, which shows details of an immobilized target-specific switch probe.

FIG. 5 depicts a magnified view of an alternative particle with respect to FIG. 4, which shows details of an indirectly immobilized target-specific switch probe.

FIG. 6 depicts an immobilized probe of FIG. 4 after the target-specific portion of the probe has been extended by a DNA polymerase in the presence of the target molecule.

FIG. 7 depicts the immobilized extended probe of FIG. 6. together with dissociated target molecule and dissociated quencher sequence, under conditions in which the reaction temperature was raised above the nucleic acid melting temperature.

FIG. 8 depicts the immobilized extended molecule of FIG. 7 under conditions in which reaction temperature has been lowered below the nucleic acid melting temperature such that the extended sequence has hybridized to the switch portion of the probe, preventing re-hybridization of the quencher portion of the probe and allowing the fluorophore to generate a detectable signal.

FIG. 9 depicts the immobilized target-specific switch probe of FIG. 4 when no target sequence is present and extension does not occur.

FIG. 10 depicts a holographically encoded particle having capture moieties immobilized to its surface.

FIG. 11a depicts single-stranded nucleic acid probes labeled with detectable tags in solution. FIG. 11b depicts hybridization of a portion of each labeled probe with a portion of a linker probe. FIG. 11c shows the labeled probe-linker probe complexes when captured by specific hybridization to the capture sequences of a holographically encoded particle.

FIG. 12a depicts an exemplary probe. FIG. 12b depicts the probe when bound to a target molecule and extended by a DNA polymerase. FIG. 12c depicts capture of a non-extended probe by a capture sequence on a particle. FIG. 12d depicts capture of an extended probe by a capture sequence on a particle.

FIG. 13 is an expanded depiction of FIG. 12c, which shows non-extended primers captured onto a holographically encoded particle by its immobilized capture moieties. FIG. 14 depicts immunoassay capture antibodies conjugated to nucleic acid sequences complementary to capture moieties immobilized on multiplex assay particles. The conjugated target-specific antibodies are shown in solution.

FIG. 15 depicts the conjugated target-specific antibodies of FIG. 14 after they have specifically bound to targets in an assay in solution.

FIG. 16 depicts the antibody-target complexes of FIG. 15 after incubation with a labeled detector antibody that specifically binds to another epitope of the target in a sandwich assay.

FIG. 17 depicts the antibody-target-labeled detector antibody complexes of FIG. 16 after incubation with a holographically encoded particle. The particle has capture moieties immobilized on its surface that are complementary to the complementary sequence conjugated to the target-specific antibody, enabling the particle to capture the assay components by specific hybridization.

### DETAILED DESCRIPTION

The methods described herein can be used, e.g., for detecting at least two target molecules in a sample (e.g., a single sample). The term “target molecule” refers to any detectable molecule. Accordingly, the methods can be used to detect a variety of target molecules, including macromolecules such as nucleic acid and protein, and smaller molecules such as carbohydrate, lipid and nucleotides. Nucleic acid targets include genomic DNA, mitochondrial DNA, messenger RNA, and replicates thereof (e.g., cDNA and amplified versions thereof).

Many of the methods described herein are suitable for use in genomic analysis and screening assays, including clinical assays. The methods can include an encoded particle system for performing multiplexed assays for proteins, peptides, nucleic acids and other molecules. The particles can include target-specific probes (e.g., probes that detect target molecules or assay components) or capture moieties that specifically bind assay reagents, such as target specific probes.

A “capture moiety” is a moiety that can specifically bind to a complementary moiety on a target-specific probe. It is used to link target-specific probes to particles. The capture moieties are generally particle-specific moieties that interact with either a specific target molecule or a complementary moiety that is physically associated with a target-specific probe. In many embodiments, a capture moieties is an oligonucleotide that has a “capture sequence.” The target-specific probe can include a complementary moiety which corresponds to a region of an oligonucleotide that is complementary to the capture sequence.

A “target specific probe” is any molecule that recognizes a target molecule with sufficient affinity and specificity to bind and distinguish the target molecule from unrelated target molecules. Examples of target specific probes include oligonucleotides and antibodies. Antibodies include full length antibodies, e.g., IgG's or IgM's as well as fragments such as Fab, Fab2, scFv, camelids, and single chain antibodies. Other binding proteins can be used such as extracellular domains of cell surface receptors and specific binding peptides. Moreover, a variety of target-specific probes in addition to antibodies, oligonucleotides and other hybridizing nucleotide sequences can be used in a similar manner. Non-limiting examples of such binding partners include proteins, protein-nucleic acids, nucleic acid aptamers, and other artificial hybrid binding partners, receptors, ligands, antigens and the like.

The particles can also be associated with target specific probes after the target-specific probes are contacted to a sample, and associated with target molecules. The particles having collected target-specific probe-target complexes are then detected.

In one aspect, ZIP codes are used to associate a set of encoded particles with unique target-specific probes. ZIP codes refer to universal capture moieties that have one or more of the following characteristics (e.g., all of the following characteristics):
the capture moieties comprise oligonucleotides made synthetically;
the capture moieties do not interact with target sequences expected in a sample;
the capture moieties of a given set have melting temps (Tₘ) within a narrow range (e.g., within 5, 4, or 3 degrees) so they will all hybridize in a narrow range of conditions;
the capture moieties are immobilized or can be immobilized to a solid substrate in an identifiable manner, e.g. a region of a microarray (identifiable by its row/column position in an array) or an encoded particle (identifiable by a code);
the complementary moiety (e.g., the complementary strand of a zip-code pair) is attached to a target specific probe, e.g. an antibody, a Scorpion probe, a hybridization probe, or an aptamer.

A multiplex specific binding assay using ZIP codes can be performed in solution. For example, each species of target-specific probe in the multiplex set is physically associated with (e.g., covalently bound to) a complement moiety. In one embodiment, after the multiplex assay is complete, the multiplex assay components or products are specifically collected (“fished out”) onto the solid substrates by specific binding of the capture moiety to the complementary moiety. The uncollected sample and assay byproducts are then washed away. Once the assay components or products are bound (e.g., hybridized) to the solid substrates by the ZIP codes, they can be evaluated, e.g., using a detection method. For example, an array can be imaged. Particles can be processed, e.g., in a fluorescent sorter. One advantage of this approach over simply immobilizing the target specific probes onto the solid substrates is that the target binding portion of the assay is performed in solution with improved kinetics and reduced steric interference. In other embodiments, the ZIP codes are used to associate target specific probes with a substrate prior to contacting a sample. Still other embodiments do not use ZIP codes.

In some embodiments, target molecules are detected using target-specific switch probes. Target-specific switch probes are capable of “switching on” or otherwise altering their signal when a target molecule is present, and therefore can be detected without a need for adding additional detection reagents to the assay vessel, allowing for a homogenous assay format. However, other types of target-specific probes are also well suited for performing homogeneous multiplexed particle-based assays. For example, it can be desirable to add such additional reagents with non-switch type probes. Target specific switch probes can be immobilized to a substrate, e.g., a particle (such as an encoded particle), or to an array, e.g., an area on array.

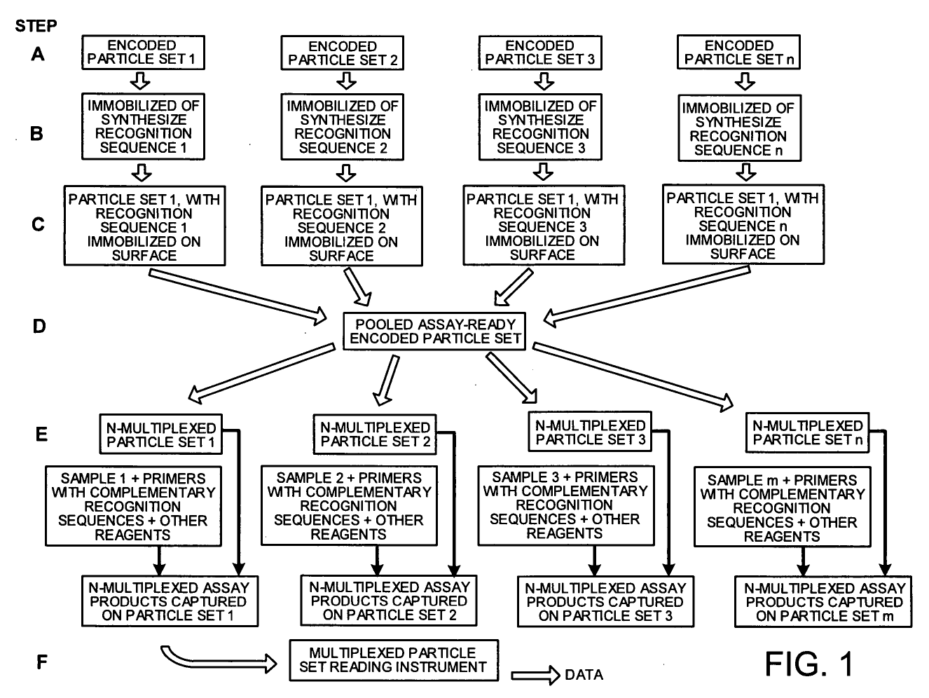

Referring to FIG. 1, the general steps A through F for preparing and performing a holographically encoded particle multiplex assay are shown in the flow chart. The number of target molecules to be assayed for is defined as n. In Step A, sets of holographically encoded glass particles 1 through n, in which the particles of each set are encoded with the same identification code, are provided and kept separate. The particles can be made of any material that can be holographically or otherwise encoded, such as by a diffraction grating, and can accept or be modified to accept attachment of a particle specific sequence, e.g., a capture sequence or a probe. Exemplary materials include glass, such as silica, phosphate glass, borosilicate glass, and other glasses; combinations of glass and other materials, such as plastics; and plastic.

In Step B each set of encoded particles is provided. Each particle of the set includes a particle specific probe (e.g., a capture moiety or a target specific probe, e.g., a target specific switch probe). A particle specific probe is typically a nucleic acid oligonucleotide that is covalently attached to the particle. The particle specific probe can include a region that is specific for a target molecule. Alternatively, the particle specific probe can include a region that is specific for a target-specific probe. In such cases, the particle specific probe itself need not include a region specific for a target molecule. For example, the particle specific probe can include a capture moiety.

In case of a particle specific probe that is a nucleotide sequence, a single-stranded nucleic acid sequence of about 10 to about 100 bases long can be used to specifically bind to its complement such that the sequence does not appreciably cross-react with other assay products or components. A particle specific probe that is a nucleotide sequence can be synthesized directly upon the glass particles, for example using phosphoramidite chemistry in a standard oligonucleotide synthesizer and initiating the synthesis on the surface of aminosilane coated particles.

In Step C the particles with immobilized particle-specific probes have been removed from the solution of capture probe or synthesis solution. This can be done by pipetting, or by filtering, centrifugation, or any other standard laboratory process for separating solid particles from liquids. The result at this step is n separate sets of encoded particles in which the particle of each set have the same code and immobilized capture moiety, but the codes and particle-specific sequences differ between the sets. In Step D these n sets are pooled together and mixed, typically in an aqueous buffer.

At Step E, aliquots of particles have been taken from the pooled set to form a plurality (m) of n-multiplexed encoded particle sets. These aliquots typically have nominally identical numbers of particles in them and nominally equal populations of each of the n species of particles as well. The distributions can deviate from the nominal conditions due to randomness and tolerances on the mixing of particles within the pool and the aliquotting process. Typically, the aliquots are sized so that they contain multiple replicates of each particle type. For example, the number of replicates in an aliquot can be from about 3 to about 5,000, from about 3 to about 10, from about 10 to about 50, from about 50 to 500. The number of replicates can be used to evaluate the statistical significance of any observation. By aliquotting replicates beyond the minimum number of particles needed to generate a valid signal the user can be assured that all target molecules will be assayed for with each n-multiplexed particle set. The number of different particles can be large, e.g., at least 10, 50, 100, 200, 250, 500, or 1000 particles, e.g., between 100-5000 or 100-100,000 thousand different particles (optionally including multiple replicates of each particle type or substantial replicates that differ only by slight differences in identifier codes or different codes that are associated with the same object in a database.

The number of samples to be assayed by the collection of identical particle sets shown here is m. Each multiplexed assay is performed in solution in a fluid-containing vessel such as a microplate well. Such a vessel would be loaded in the proper order with a sample to be assayed, and with the other reagents such as enzymes, antibodies, labeling reagents, buffers, etc. After one or more incubations, and any required washing, thermal cycling, or agitation steps, the multiplex assay is complete. The assay products can have fluorescent or other labels incorporated into them approximately proportionally to target molecule the amount or concentration of each target molecule in the sample. In some embodiments, such assay steps are performed prior to the addition of the encoded particles. Each assay product has the complement to a capture moiety incorporated into it so that the target molecule can be specifically captured by an encoded particle. In the example shown, the assay is a multiplexed primer extension genotyping assay. Each primer sequence has on its 5'end, a sequence complementary to capture sequences. Other types of assays would have the complement to the capture sequence incorporated into a specific binding pair member, e.g., as illustrated in later figures.

After completion of the multiplex assay, the aliquots of particles are each utilized to specifically capture the products of an n-multiplexed assay from each of the m samples. The capture sequence on each of n type of particle hybridizes with the complements to said sequence that are incorporated into each of n types of assay products. After another incubation period to facilitate that hybridization and possibly a wash, the particle set with the labeled assayed target molecules bound to each particle is removed from the assay vessel and transferred to a reading instrument at Step F. The reading instrument reads the particle identifier and signal associated with one or more assay labels from each particle. Signals from replicate particles with the same codes and coatings can be consolidated in the instrument or in a downstream data processing computer. For example, it is possible to average signals from particles with the same codes and to perform other statistics, e.g., to determine standard deviation etc.

The encoded particle multiplex assay process shown in FIG. 1 can be applied to a variety of specific binding assays. An example is use in a primer extension genotyping assay. As such, capture sequences can be used to specifically capture any type of binding pair member. Non-limiting examples of assays adaptable to the present technology include SNP detection, DNA sequencing, expression profiling, antigen detection, and antibody screening.

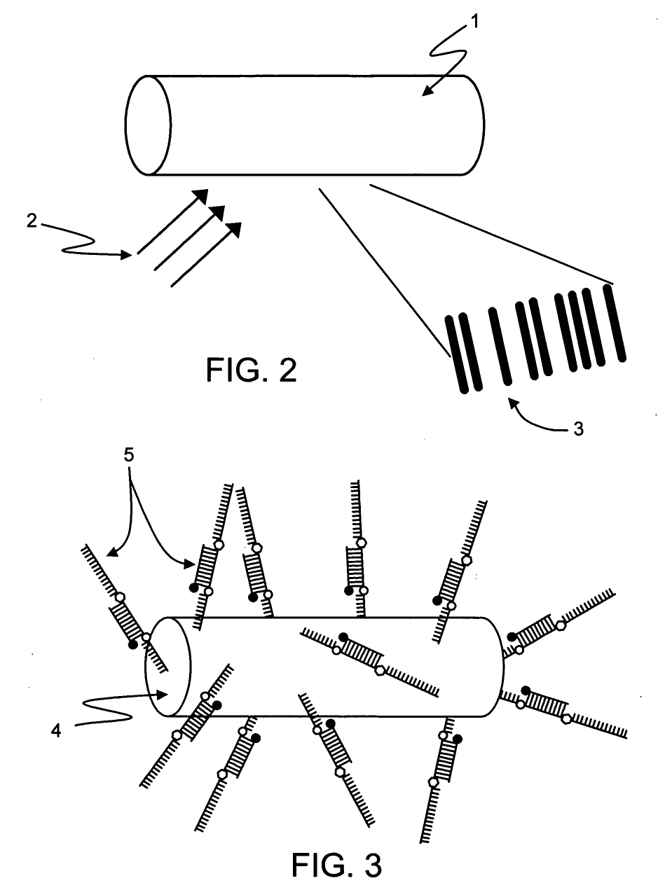

FIG. 2 depicts a holographically encoded particle. A “holographically encoded particle”is a particle that has a hologram or diffraction grating that functions an identifier. An example of an identifier is a barcode. The holographically encoded particle 1 is interrogated by a beam of parallel light 2 at controlled wavelength, polarization and incidence angle. For example, the beam can be a laser beam at 532 nm and the particle can be cylindrical and oriented to the beam in a transparent flow capillary or by lying in an oriented groove in a grooved particle-reading plate. Such a cylindrical particle can, for example, be made from a length of glass fiber, with a diameter between about 10 μm and 100 μm and a length between about 25 μm and 250 μm. In an embodiment, the particles are 28 μm in diameter and 200 μm long. The holographic image 3, shown here as a barcode, is projected out from the particle at an orientation and image divergence set by the hologram recording conditions. In an embodiment, the hologram image diverges as it projects away from the particle such that it is several mm long at a distance of about 10 to about 100 mm away from the particle. This allows the barcode to be read easily by a simple, inexpensive low-resolution imaging array such as a charge-coupled device (CCD).

It is possible to evaluate a particle using a laser that interrogates the hologram. In an embodiment, the laser can also excite a fluorophore assay label such as cyanine 3 or tetramethyl rhodamine simultaneously. The fluorescence signal is captured by an objective lens with an optical axis non-coincident to the excitation beam or the hologram. One can select an appropriate fluorescent label, for example, based on the emission, absorption and hydrophobic/hydrophilic properties desired, photostability and quantum yield.

By way of example, coding can be implemented using a digital format, e.g., bits. The bits can be optically encoded, e.g., holographically encoded. The coding can include a variety of syntax information, e.g., start and stop bits and bits for error checking, such as a check sum or cyclic redundancy check. The error check portion ensures that the code which is obtained from the particle is accurate. Use of a large number of bits increases statistical accuracy in the code readout and decreases erroneous code. The code section may be broken up into one or more groups of bits, for example, three bit groups. The code section may provide information about the particle itself or the item attached to the particle or how the particle is to be used, or other information. For example, the code section may contain information regarding “identifying numbers”, such as: lot number, quality control number, model number, serial number, inventory control number; the second bit group may contain “type” information, such as: chemical or cell type, experiment type, item type, animal type; and the third bit group may contain “date” information, such as: manufactured date, experiment date, creation date, initial tracking date. Any other bit groups, number of bit groups, or size of bit groups may be used if desired. Also, additional error or fault checking can be used if desired. In a simple embodiment, the coding includes merely a serial number. The serial number can be used to reference other information, e.g., information stored in a database.

In certain cases, a holographically encoded particle or other particle can be used in connection with a plurality of different target-specific probes, e.g., target specific switch probes. The probes can be specific to different target molecules or can be specific to different regions of the same target molecule. In such cases, different labels that have sufficiently different spectra are used. For example, the selected labels can have similar or overlapping excitation spectra but different emission spectra, such that the dyes are spectrally distinct. When differentiation between two or more labels is accomplished by instrumentation, a variety of filters and diffraction gratings are commercially available to allow the respective emission maxima to be independently detected. When two or more dyes are selected that possess relatively small differences in emission maxima, instrumental discrimination can be enhanced by ensuring that the emission spectra of the two or more labels have similar integrated amplitudes and similar emission peak widths and that the instrumental system's optical throughput will be equivalent across the emission peak widths of the respective labels.

The exemplary encoded particle multiplex assay process in FIG. 1 can be applied to a variety of specific binding assays. In one embodiment, the binding assay includes target-specific switch probes that are immobilized to the encoded particles and measurement of multiple nucleic acid targets in each sample. As used herein, the term “target-specific switch probe”means a molecule or complex of molecules capable of selectively binding to a specific nucleic acid sequence, and in so doing, becomes capable of producing a detectable signal indicative of presence or absence of a defined target molecule. Exemplary target-specific switch probes are described in Whitcombe, et al. (1999) Nature Biotech 17, 804-807.

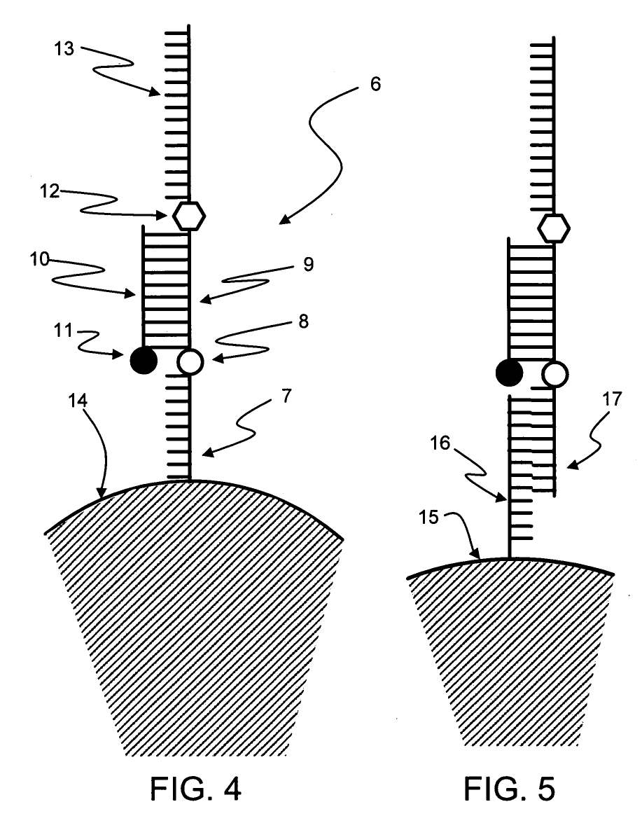

FIG. 3 depicts the encoded particle 4 of FIG. 2 with target-specific switch probes 5 attached to its surface, for example from the in-situ synthesis process noted in FIG. 1 Steps B and C. Exemplary particles can be cylindrical in shape or any other shape, e.g., spherical. Particles can be solid or porous.

FIG. 4 depicts an enlarged view of an exemplary target-specific switch probe immobilized on a section of the surface of the encoded particle of FIG. 3. An exemplary probe's constituent parts are separately called out. The probe 6 is immobilized onto the surface of a particle 14. The probe consists of a target-specific primer sequence 13, the sequence to be extended by a polymerase enzyme in the presence of a complementary target sequence in the sample. The primer sequence 13 is connected to and isolated from a probe sequence 9 by a blocker 12, the blocker comprising a hexethylene glycol (HEG) monomer, for example. A fluorophore 8 is incorporated at the other end of the probe sequence.

Additional and optional features of a target-specific switch probe are described herein below. The “switch” feature of this exemplary probe is imparted by the ability of a primer extension product (arising from the presence of the target sequence) to bind to the switch portion of the probe, thereby displacing the quench portion of the probe and allowing detection of the fluorescent moiety. In the absence of a primer extension product (arising from the absence of the target sequence), the quench portion of the probe remains bound to the switch portion of the probe. In this configuration, the fluorescent moiety remains quenched and no substantial fluorescence signal is detectable. An example of this type of target-specific switch probe is described in Whitcombe et al. Nature Biotechnology (1999) 17:804 and U.S. Pat. No. 6,326,145.

A linker 7 attaches the entire probe construct to the surface of the particle. A suitable linker generally has a length and flexible that prevents unwanted interaction (for example, steric interference) between the particle and the immobilization chemistry from influencing subsequent hybridization reactions in the probe sequence. An exemplary linker, shown in FIG. 5, is a sequence of single-stranded nucleic acid 17, which can be captured by a fairly short (10-30 mer) oligonucleotide 16 synthesized on the surface of the particle 15. This embodiment allows the relatively simple synthesis of a short oligo on the particle by conventional means and the specific capture and immobilization of the target-specific switch probe through a robust and flexible linker. Such synthesis typically is initiated from an aminosilane coated particle using conventional phosphoramidite oligonucleotide synthesis chemistry. Alternatively, pre-synthesized oligonucleotides can be immobilized upon the particle surface to accomplish the same end.

Referring again to FIG. 4, prior to the assay a quencher sequence 10 with a quencher molecule 11 is hybridized to the probe sequence of the molecule. An exemplary quencher is DABCYL, appropriate for use with exemplary fluorophores such as the various cyanine dyes such as cyanine 3. When the primer sequence 13 is not extended the quencher 11 is located within nanometer proximity to the fluorophore 8 preventing the fluorophore from generating a signal. In an alternative embodiment, the quencher can be embodied as a plurality of quenching molecules, such as a dendrimer structure, in order to improve assay signal-to-noise ratio by more thoroughly quenching the fluorophore than a single quencher molecule.

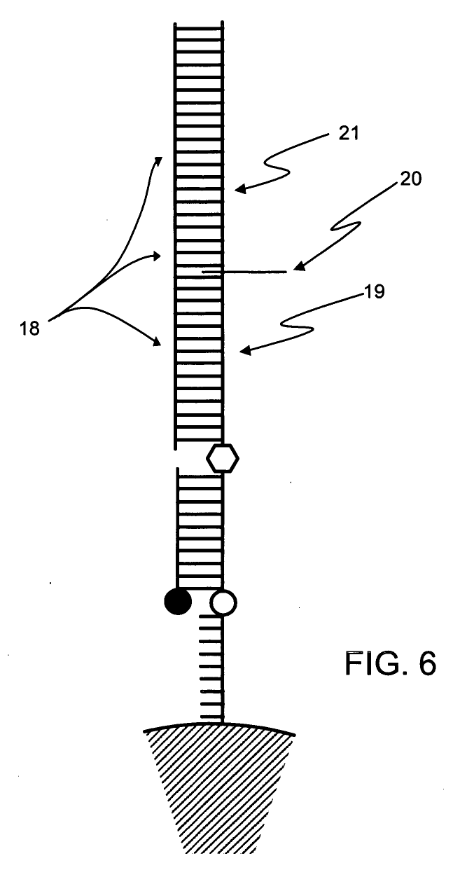

FIG. 6 shows the target-specific switch probe of FIG. 4 following primer extension. When a complementary target sequence 18 is present in the sample, the primer 19 is extended by a polymerase enzyme to form an extended sequence 21. In the figure the dotted line 20 marks the boundary between the original primer sequence and the extended sequence. If the target sequence is not present, or not present in sufficient quantity, in the sample the primer is not extended. Most often, the target sequences present have been previously amplified and increased in abundance in the sample by a process such as multiplex PCR prior to the assay.

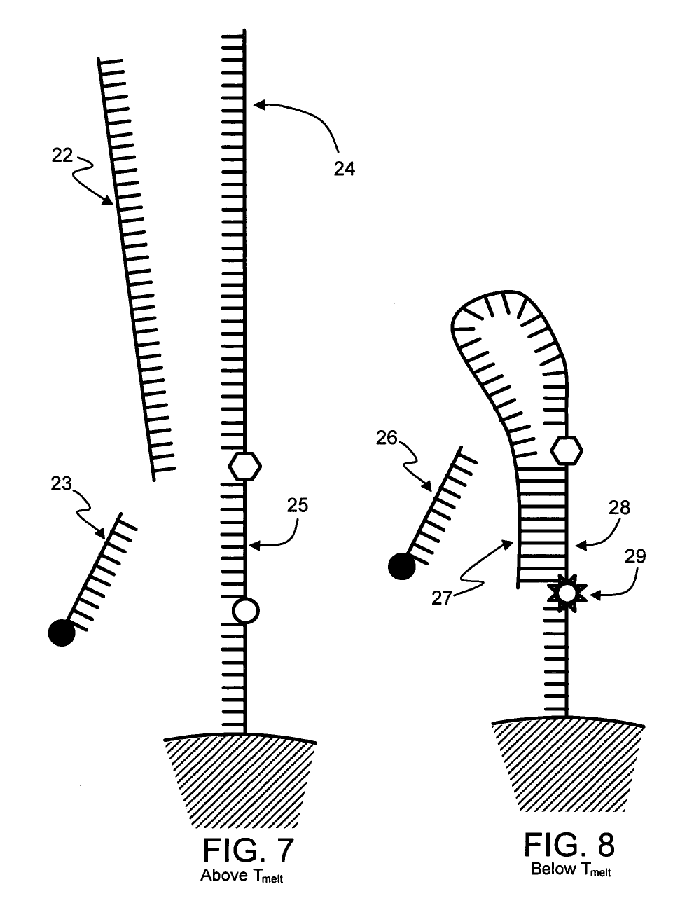

FIG. 7 depicts the extended probe of FIG. 5 where the ambient temperature has been increased to or above the melting temperature of the sequences in the probe and extended sequence. Under these conditions the target 22 de-hybridizes from the extended primer 24 and the quencher sequence 23 de-hybridizes and dissociates from the probe sequence 25.

In FIG. 8, the temperature has been reduced to a point below the melting temperature allowing hybridization to occur. The probe sequence 28 had previously been chosen to be complementary to the reverse-order of the end of the extended sequence 27. Further, the sequences of both the probe sequence 28 and the end of the extended sequence 27 have been chosen such that the thermodynamics of hybridizing the extended sequence to the probe is preferable to those of the quencher sequence 26. For example, the quencher sequence can have a single-base mismatch to the probe sequence, where the end of the extension product does not. In this case the end of the extension sequence displaces the quencher, allowing the fluorophore 29 to produce a signal when excited.

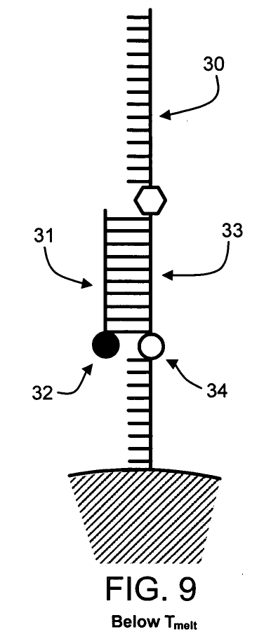

FIG. 9 depicts the target-specific switch probe following the processes depicted in FIG. 5 to 7, only in the absence of a target molecule. After melting, the temperature is brought below the melting temperature the quencher sequence 31 re-hybridizes to the probe sequence 33, quenching the fluorophore 34 by the proximity to the quencher molecule 32. Thus, when the target sequence is not available to allow primer extension, the fluorescence remains quenched and no or minimal signal is generated.

The reagents described herein (e.g., particles, probes, and capture sequences) can be provided as assay kits and systems for performing particle-based multiplexed assays employing target-specific switch probes. In particular, kits for performing SNP assays using primer extension are provided. Such kits can include multiple probes, each having a different fluorescent label, if desired. For example, three or more, four or more, or five or more probes having different fluorescent labels can be provided.

A particle can have any geometry or physiochemical characteristics so long as encoded information can be detected and probes can be attached. A particle can be encoded by any detectable means, such as by holographic encoding, by a fluorescence property, color, shape, size, light emission, quantum dot emission and the like. As is described in detail below, in one embodiment, holographically encoded particles are used. Several particle-based assay systems have been previously described. Chandler et al. (U.S. Pat. No. 5,981,180) describes a particle-based system in which different particle types are encoded by mixtures of various proportions of two or more fluorescent dyes impregnated into polymer particles. Applications of the Chandler system to non-homogeneous primer extension SNP assays are described (U.S. Pat. Nos. 6,004,744 and 6,013,431). Soini (U.S. Pat. No. 5,028,545) describes a particle-based multiplexed assay system that employs time-resolved fluorescence for particle identification. Fulwyler (U.S. Pat. No. 4,499,052) describes an exemplary method for using particle distinguished by color and/or size. US 2004-017926, 2004-0132205, 2004-0130786, 2004-0130761, 2004-30126875, 2004-0125424, and 2004-0075907 describe exemplary particles encoded by holographic barcodes. Exemplary uses described for the particle-based methods referenced above include nucleic acid hybridization assays and immunoassays. The particles can also be used for homogeneous primer extension SNP assays. In other cases, the particles are not used for homogenous primer extension SNP assays.

An apparatus for detecting a target-specific switch probe can include a means for exciting target-specific switch probes, and means for detecting emission from the probes. For example, the apparatus can include a light source, such as a laser or lamp, and can include a detector such as a CCD. The apparatus can include a flow path, e.g., for channeling particles past the light source. The path can be designed such that a single particle is in the excitation and/or detection zone at one time. A fluorescent activated cell sorter (FACS) can be used in some implementations.

In certain embodiments, target-specific switch probes can be used in combination with a nucleic acid microarray. For example, target-specific switch probes can be associated with an array, before, during, or after, the target-specific switch probes are contacted to a sample. The substrate can include different capture moieties, e.g., a unique capture moiety at each address to recruit a complementary moiety associated with a unique target specific probe, e.g., a target specific switch probe.

Solid substrates can be porous or non-porous. A substrate material can be selected and/or optimized to be compatible with the spot size (e.g., density) required and the application. In some embodiments, the substrate is non-porous, e.g., impermeable. Exemplary solid substrates include: mass spectroscopy plates (e.g., for MALDI), glass (e.g., functionalized glass, a glass slide, porous silicate glass, a single crystal silicon, quartz, UV-transparent quartz glass), plastics and polymers (e.g., polystyrene, polypropylene, polyvinylidene difluoride, poly-tetrafluoroethylene, polycarbonate, PDMS, acrylic), metal coated substrates (e.g., gold) , silicon substrates, latex, membranes (e.g., nitrocellulose, nylon), a glass slide suitable for surface plasmon resonance (SPR). In other embodiments, the solid substrate is porous, e.g., a gel or matrix. Potentially useful porous solid substrates include: agarose gels, acrylamide gels, sintered glass, dextran, meshed polymers (e.g., macroporous crosslinked dextran, sephacryl, and sepharose), and so forth.

The substrate can be opaque, translucent, or transparent. The addresses can be distributed, on the substrate in one dimension, e.g., a linear array; in two dimensions, e.g., a planar array; or in three dimensions, e.g., a three dimensional array. The solid substrate may be of any convenient shape or form, e.g., square, rectangular, ovoid, or circular. The substrate can contain at least 1, 10, 100, 10³, 10⁴, 10⁵, 10⁶, 10⁷, or more addresses per cm². The center to center distance can be 5 mm, 1 mm, 100 μm, 10 μm, 1 μm, 100 nm or less.

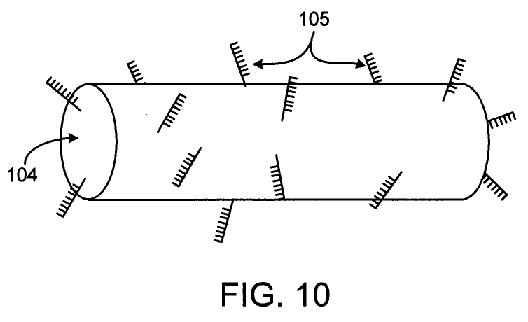

FIG. 10 depicts an exemplary cylindrical encoded particle 104 of FIG. 2 with capture sequences 105 attached to or immobilized to it, e.g., on its surface. The molecule containing the capture sequences can be attached or immobilized by covalent or non-covalent interaction, absorption, dissolution, surface adsorption, and the like. The particle can be, for example, an optically encoded particle such as a holographically encoded particle.

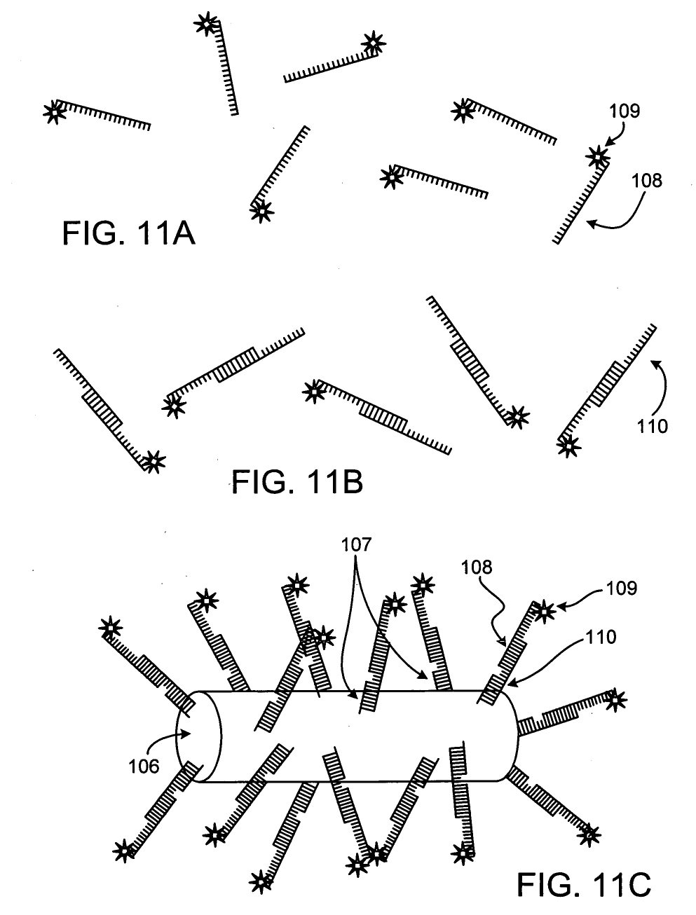

FIG. 11a depicts an exemplary nucleic acid assay. In FIG. 11a, target sequences 108 from the assay sample are shown labeled with a fluorescent label molecule 109. Such labeled target sequences can be from a reverse-transcription RNA transcription assay, for example, where one or more fluorophore molecules are enzymatically incorporated into cDNA complements transcribed from RNA derived from a biological sample. In FIG. 11b the labeled target molecules have hybridized with a linker sequence 110. In this case, the linker sequence is complementary to the target on one end and complementary to the immobilized capture sequence on the other end. In FIG. 11c the free end of the linker sequence of the complex of FIG. 11b has hybridized to the capture sequence 107 that is immobilized on the holographically encoded particle 106. With capture and linker sequences different for each nucleic acid target, the step illustrated in FIG. 11c captures signal-producing assay products for each sample sequence on particles with different identification codes, enabling multiplex nucleic acid assays. For example, the particles can be analyzed, e.g., using a scanner that can detect the holographic code of the particle and signal.

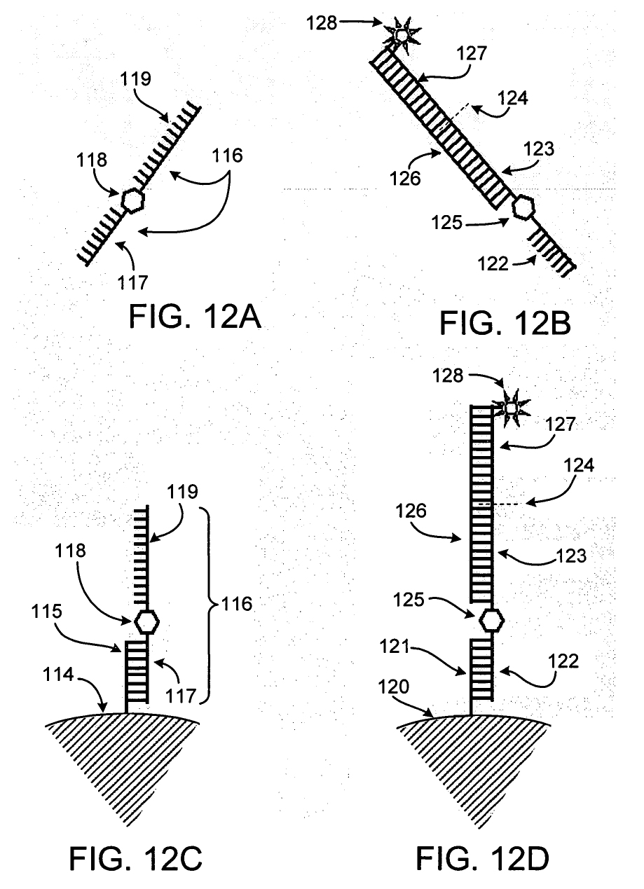

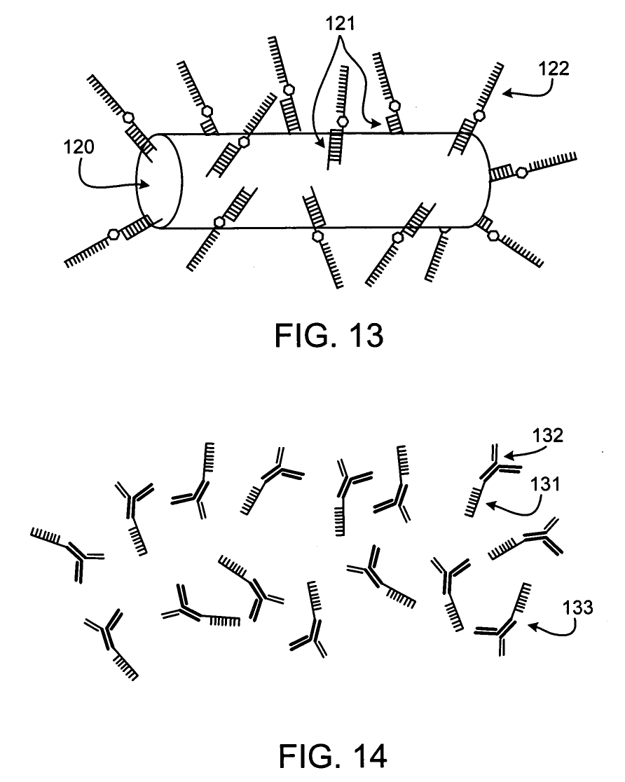

FIG. 12 depicts exemplary mechanisms for primer-extension SNP assays that can be used. In FIG. 11a, a capture-encoded primer 116 is composed of a capture sequence complement 117, a blocker 118 and a primer sequence 119. When incubated in the presence of a target sequence from the sample that is complementary to the primer, the primer can be extended by a polymerase reaction as shown in FIG. 12b. A target sequence 126 hybridizes to the primer 123, and the primer is extended in double-stranded form past its original end denoted by the dotted line 124. In one form of genotyping assay, the extension sequence 127 is terminated by a fluorescently labeled terminator 128, wherein the different terminating nucleotides are labeled with different color fluorophores. In these assays the color of the fluorophore indicates the type of terminating nucleotide, hence the genotype at that location. FIG. 12c shows a non-extended primer 16 captured on the surface of a holographically encoded particle 14, wherein the primer's complementary sequence 17 is hybridized to the particle's capture sequence 15. FIG. 12d depicts capture of the extended primer of 5b by the same mechanism. FIG. 13 depicts schematically an exemplary particle 120 having captured a plurality of non-extended primers 122 on its capture sequences 121; such a particle represents a negative result for sequences complementary to the primer in a sample.

FIG. 14 through FIG. 17 depict an exemplary embodiment that uses antibodies or other proteins that function as specific binding agents. Binding of a target molecule to a target specific probe such as an antibody can be detected, for example, using sandwich immunoassays. FIG. 14 depicts antibody complexes 133 in solution. Each complex comprises an antibody 132 and a nucleotide sequence 131 attached or conjugated to the antibody. The nucleotide sequence is complementary to a capture moiety on an encoded particle and therefore can function as a complementary moiety for recruiting the target specific probe to a substrate, e.g., a holographically encoded particle.

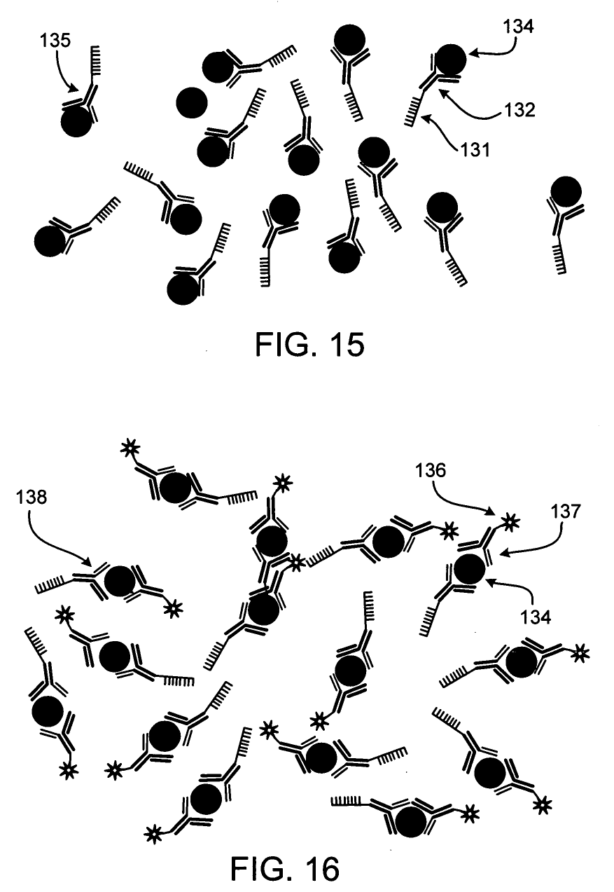

FIG. 15 depicts the antibody complexes of FIG. 7 after incubation with a sample wherein the sample contains protein antigens to which the antibodies specifically bind. In FIG. 15, antibodies 132 are shown binding to proteins 134 to form complexes 135. These complexes are still conjugated to the nucleotide sequence 131 that functions as a complementary moiety. Although in this example, the antigen is a protein, it is understood that antibodies can be raised against a variety of molecules, including small molecules such as cAMP. Thus, a target molecule detected using an antibody in the methods described herein can be any molecule against which an antibody can be raised. An antibody useful in a method described herein alternatively can be selective for an entity bound to a target. As such, a sample to be analyzed using a method described herein can be modified, if desired, prior to analysis, to attach or incorporate a particular antigen to a target molecule.

In FIG. 16 the complexes shown in FIG. 15 are subsequently incubated with a labeled detector antibody to form labeled immunoassay complexes 138. The detector antibody can specifically bind to the captured protein 134 and includes a label such as a fluorophore 136. The target-specific antibody and the detector antibody can bind to non-overlapping epitopes of the protein to enhance the specificity of the assay.

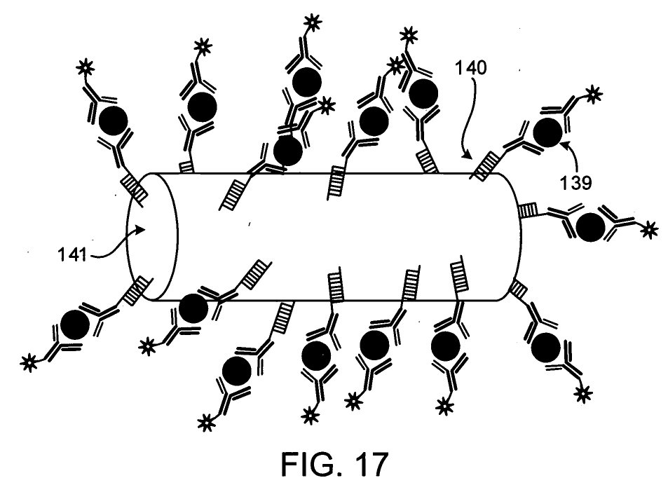

In FIG. 17 the assay products 139 of FIG. 16 are captured onto holographically encoded particles 141. During an incubation with the particles, the sequences conjugated to the target-specific antibody hybridize to capture sequences immobilized on the particle, forming a specifically bound double-stranded sequence of nucleic acid 140. For multiplexed applications of the present method, a different set of sequences and antibodies are selected for each target molecule to be detected. The assay is performed in multiples, and the data for each target molecule is resolved by reading the particle identification from its holographic code.

A variety of labels in addition to fluorescent moieties can be used. Non-limiting examples of such labels include fluorescent dye-containing particles, such as nanoparticles, radioactive moieties and the like.

A manufacturer and marketer of multiplex assay consumables may manufacture, procure, inventory, track and offer for sale thousands or tens of thousands of individual assay components, due to the wide variety of assay chemistries, target molecule types, and specific target molecules. For example, to substrate 1,000 different SNP target molecules using two different assay chemistries would require 2,000 different encoded particle types to be available. If, in addition, 1,000 different immunoassay target molecules were substrateed a total of 3,000 different encoded particle types would be required. Procuring and keeping in inventory such an extensive collection presents significant logistical challenges and expense.

Utilizing the universal capture sequences such as ZIP codes enables reducing the number of different types of encoded particles required. In the research and diagnostic fields where the automation and robustness advantages of particle multiplexing are most pronounced. The degree of multiplexing n rarely exceeds 200, and only very rarely exceeds 300. By choosing a constraint of nₘₐₓᵢₘᵤₘ=300 provider of particle multiplex assays needs only to offer 300 different particle types, with 300 holographic codes and 300 corresponding universal capture sequences. From this library of particles and unlimited number of multiplex assay kits can be constructed utilizing multiple assay chemistries and target molecule types. This smaller library of particles requires significantly less investment to generate and maintain.

A variety of SNP detection methods can be similarly used, e.g., with encoded particles, e.g., optically encoded, e.g., holographically encoded particles or with the ZIP codes described herein. Exemplary SNP detection methods include TAQMAN™, in which a complementary probe is cleaved during PCR to release probe-specific fluorescence; INVADER™ squared, in which a triplex structure formed between the target sequence, invader probe and primary probe is cleaved by a flap endonuclease; single base extension, in which fluorescently labeled ddNTPs are incorporated into an extension primer; SNaPIT™, in which a diagnostic PCR primer is annealed closed to the SNP and dUTP is incorporated in place of dTTP; UDG digestion results in different length fragments representing the different alleles; and OLA™, in which a fluorescently labeled allele-specific probe is hybridized to the target DNA and ligated to a generic biotin-labelled probe.

The following publications and patent documents are incorporated herein by reference in their entirety. Published U.S. Patent Application references include 2004-0179267; 2004-0132205; 2004-0130786; 2004-0130761; 2004-0126875; 2004-0125424; 2004-0075907 . U.S. Patent references include U.S. Pat. Nos. 4,499,052; 5,028,545; 5,981,180; 5,432,049; 5,888,818; 6,004,744; 6,013,431; 6,300,063; and 6,326,145.

Non-patent reference publications include Hirschorn et al., “SBE-TAGS: An array-based method for efficient single-nucleotide polymorphism genotyping”, PNAS 97-22, Oct. 24, 2000 p. 12164-12169; Pastinen et al., “A system for specific, high-throughput genotyping by allele-specific primer extension on microarrays”, Genome Research 10, Aug. 7, 2000, p. 1031-1042; Chang et al., “Microarray-based detection of select cardiovascular disease markers”, BioTechniqes 29-5, November 2000, p. 936-944; Schena et al., “Quantitative monitoring of gene expression patterns with a complementary DNA microarray”, Science 270, Oct. 20, 1995 p. 467-470; Fodor et al., “Light-directed, spatially addressable parallel chemical synthesis”, Science, Feb. 15, 1991 p. 767-773; Haab et al., “Protein microarrays for highly parallel detection and quantitation of specific proteins and antibodies in complex solutions”, Genome Biology 2001 2(2):research 0004.1-0004.13; Tm Bioscience Technical Bulletin—401 “Tm-100 Universal Sequence Set”, Mar. 8, 2002; Tm Bioscience Technical Bulletin—403 “Genotyping on the Tm/Luminex Universal Array Platform Using Primer Extension Chemistry”, Mar. 13, 2002. Exemplary Molecular protocols can be found, e.g., in Ausubel, F. et al., eds. (1999) Current Protocols in Molecular Biology, J. Wiley: New York).

Other embodiments are within the following claims.

What is claimed is:

1. A solid substrate having attached thereto a target-specific switch probe.

2. The substrate of claim 1, wherein the substrate is a particle.

3. The substrate of claim 2, wherein the particle is holographically encoded.

4. The substrate of claim 1, wherein the substrate comprises a planar surface that comprises a plurality of positionally distinguishable addresses and wherein the target-specific switch probe is attached to at least one of the addresses.

5. The substrate of claim 4, wherein each of the plurality of positionally distinguishable addresses comprises a different target-specific switch probe.

6. The substrate of claim 1, wherein the target specific switch probe comprises a first strand that comprise a fluorescer, a probe region, a blocker, and a primer, and a second strand that comprises a quencher that is attached to quencher stand that hybridizes to the probe region unless the primer is extended, and wherein the primer is specific for a target nucleic acid sequence and is extendable to include a sequence complementary to the probe region.

7. A collection of particles wherein each particle is a substrate according to claim 2, wherein the collection includes particles having target-specific switch probes specific for different target sequences.

8. A collection of particles wherein each particle is a substrate according to claim 4, wherein the collection includes particles having target-specific switch probes specific for different targets and particles having the same target-specific switch probe have the same optical coding.

9. A method of evaluating a plurality of target molecules, the method comprising:
providing (i) a plurality of target-specific switch probes specific for different target molecules, wherein the target-specific switch probes are physically associated with a substrate and (ii) a sample;
detecting signals associated with each of the different target specific switch probes, thereby detecting target molecules based on interaction between the target-specific switch probes and the respective target molecules for which they are specific.

10. The method of claim 9 wherein the substrate comprises an array having a plurality of positionally distinguishable areas, and each target-specific switch probe is physically associated with a different area.

11. The method of claim 9 wherein the substrate is an encoded particle.

12. The method of claim 11 wherein the substrate is an holographically encoded particle.

13. A method of evaluating a plurality of target molecules, the method comprising:
providing a mixture comprising: (i) encoded particles, wherein each particle having a particular code includes a unique target-specific switch probe for detecting an target molecule, and (ii) a sample;
maintaining the mixture under conditions that permit probes of the particles to interact with target molecules in the sample material; and
detecting signals associated with each of the encoded particles, thereby detecting interactions between the unique probe of each of the encoded particles and the target molecule that it detects.

14. The method of claim 13 wherein the encoded particles are holographically encoded.

15. The method of claim 13 wherein the target specific switch probe is physically associated with the particle by binding of a capture moiety to a complementary moiety covalently attached to the target specific switch probe.

16. The method of claim 13 wherein the mixture includes multiple copies of at least some the encoded particles.

17. The method of claim 13 wherein the encoded particles comprises particles having at least 100 different codes.

18. The method of claim 13 wherein the target specific switch probe comprises a fluorescer and a quencher, and the distance between the fluorescer and the quencher is increased when the target specific switch probe hybridizes to the target.

19. The method of claim 13 wherein the target specific switch probe comprises a first strand that comprise a fluorescer, a probe region, a blocker, and a primer, and a second strand that comprises a quencher that is attached to quencher stand that hybridizes to the probe region unless the primer is extended, and wherein the primer is specific for a target nucleic acid sequence and is extendable to include a sequence complementary to the probe region.

20. A particle that includes holographically coded information that identifies the particle and a capture moiety, comprising an oligonucleotide, and a target-specific probe, wherein the target-specific probe is associated with the particle by hybridization of a region of the oligonucleotide to a complementary region of the target-specific probe.

21. A set of particles, wherein each particle includes holographically coded information that identifies the particle and a unique capture moiety identifiable by the holographically coded information, wherein (i) the unique capture moiety specifically interacts with a complementary moiety that can be attached to a target specific probe, (ii) the unique capture moiety does not substantially interact with the sample of interest, and (iii) the unique capture moiety does not specifically interacts with the other complementary moieties of other particles in the set.

22. A method of evaluating a plurality of target molecules, the method comprising:
providing a mixture comprising: (i) holographically encoded particles, wherein each particle having a particular code includes a unique target specific probe for detecting a target molecule, wherein the target specific probe is associated with the particle by a capture moiety covalently attached to the particle and a complementary moiety covalently attached to the target specific probe, and (ii) sample;
maintaining the mixture under conditions that permit probes of the particles to interact with target molecules in the sample to form encoded particle-target molecule complexes; and
detecting signals associated with each of the encoded particle-target molecule complexes, thereby detecting interactions between the unique target-specific probe of each of the encoded particles and the target molecule that it detects.

23. A method of evaluating a plurality of target molecules, the method comprising:
providing a mixture comprising: (i) unique target-specific probes for detecting different target molecules, and (ii) sample material;
maintaining the mixture under conditions that permit the unique target-specific probes to interact with target molecules in the sample material;
associating the unique probes with holographically encoded particles, such that each encoded particle specifically associates with replicates of a particular unique target-specific probe; and
detecting signals associated with each of the encoded particles, thereby detecting interactions between the unique probe of each of the encoded particles and the target molecule that it detects.

24. The method of claim 23 wherein each encoded particle specifically associates with a particular unique probe by interaction between a capture moiety covalently attached to each encoded particle and a complementary moiety covalently attached to a target-specific probe.

25. The method of claim 24 wherein the capture moiety comprises an oligonucleotide with a region that is complementary to a sequence that constitutes the complementary moiety of the target specific probe.

26. The method of claim 22 wherein the mixture includes multiple copies of at least some the encoded particles.

27. The method of claim 23 wherein the signals are generated by fluorescent nucleotides incorporated during DNA replication by extension of the unique probes.

28. The method of claim 23 further comprising amplifying nucleic acids in the mixture using the target specific probes as primers for extension of target nucleic acid molecules.

29. The method of claim 23 wherein the target-specific probe is a target specific switch probe.

30. A method for interrogating nucleic acid sequences in a sample, the method comprising:
(a) providing a plurality of encoded particles, wherein each particle having a given code is associated with or can be associated with a target-specific probe;
(b) contacting a sample to the target specific probes;
(c) maintaining the mixture under conditions in which the target specific probes interact with target nucleic acids in the sample, if present;
and
(d) associating each of the encoded particles with its respective target-specific probe either before step (b), after (b), or after (c).
(e) evaluating the interactions by detecting the code from each encoded particle and associated information about interaction with the target nucleic acids.

31. The method of claim 30 wherein each encoded particle specifically associates with a particular target-specific probe by interaction between a capture moiety covalently attached to each encoded particle and a complementary moiety covalently attached to a target-specific probe.
